SEQUENCE LISTING (20 sequences)

SEQ ID NO: 1 - protein, 14 aa, Mus musculus
VSDRDVLPGTCVWS

SEQ ID NO: 2 - protein, 14 aa, Mus musculus
IVFAEMPCGRLHLQ

SEQ ID NO: 3 - protein, 14 aa, Mus musculus
LQLRDAAPGGAIVS

SEQ ID NO: 4 - protein, 14 aa, Mus musculus
FCEEFQVPGRGELH

SEQ ID NO: 5 - protein, 14 aa, Artificial Sequence
  TRAM BB loop inhibitor peptide position 7 Pro/His mutant
IVFAEMHCGRLHLQ

SEQ ID NO: 6 - protein, 14 aa, Artificial Sequence
  TRAM BB loop inhibitor peptide position 8 Cys/His mutant
IVFAEMPHGRLHLQ

SEQ ID NO: 7 - protein, 14 aa, Artificial Sequence
  TRAM BB loop inhibitor peptide position 5 Glu/Ala mutant
IVFAAMPCGRLHLQ

SEQ ID NO: 8 - protein, 14 aa, Mus musculus
KYKAMKKDFPSILR

SEQ ID NO: 9 - protein, 14 aa, Mus musculus
LHKRDFVPGKWIID

SEQ ID NO: 10 - protein, 14 aa, Mus musculus
LHYRDFIPGVAIAA

SEQ ID NO: 11 - protein, 14 aa, Mus musculus
LHERNFVPGKSIVE

SEQ ID NO: 12 - protein, 16 aa, Drosophila melanogaster
RQIKIWFQNRRMKWKK

SEQ ID NO: 13 - protein, 30 aa, Artificial Sequence
  MyD88 blocking peptide
RQIKIWFQNRRMKWKKVSDRDVLPGTCVWS

SEQ ID NO: 14 - protein, 30 aa, Artificial Sequence
  TRAM blocking peptide
RQIKIWFQNRRMKWKKIVFAEMPCGRLHLQ

SEQ ID NO: 15 - protein, 30 aa, Artificial Sequence
  TIRAP blocking peptide
RQIKIWFQNRRMKWKKLQLRDAAPGGAIVS

SEQ ID NO: 16 - protein, 30 aa, Artificial Sequence
  TRIF blocking peptide
RQIKIWFQNRRMKWKKFCEEFQVPGRGELH

SEQ ID NO: 17 - protein, 30 aa, Artificial Sequence
  TLR1/6 blocking peptide
RQIKIWFQNRRMKWKKLHERNFVPGKSIVE

SEQ ID NO: 18 - protein, 30 aa, Artificial Sequence
  TLR2 blocking peptide
RQIKIWFQNRRMKWKKLHKRDFVPGKWIID

SEQ ID NO: 19 - protein, 30 aa, Artificial Sequence
  TLR4 blocking peptide
RQIKIWFQNRRMKWKKLHYRDFIPGVAIAA

SEQ ID NO: 20 - protein, 30 aa, Artificial Sequence
  Control peptide - BLAST generated
RQIKIWFQNRRMKWKKSLHGRGDPMEAFII

(19) United States
(12) Patent Application Publication
(10) Pub. No.: US 2010/0069297 A1
(43) Pub. Date: Mar. 18, 2010

(54) Selective Inhibition of TLR4 Signaling

(76) Inventors: Mathew J Fenton, Potomac, MD (US); Stefanie N. Vogel, Columbia, MD (US); Vladimir Toshchakov, Owings Mils, MA (US)

Correspondence Address: Elizabeth Hart-Wells;University of Maryland Baltimore, 660 W Redwood Street Rm 021, Baltimore, MD, 21201, US

(21) Appl. No.: 11/918,505

(22) PCT Filed: Apr. 14, 2006
(86) PCT No.: PCT/US2006/014243

§ 371 Date: Oct. 12, 2007

Related U.S. Application Data
(60) Provisional application No. 60/672,133, filed on Apr. 15, 2005.

(51) Int. Cl. A61K 38/16; C07K 2/00; C07K 7/08; C07K 14/00; C12N 5/00; A61K 38/10; A61P 29/00

(52) U.S. Cl. 514/12; 530/300; 530/327; 530/324; 435/375; 514/14

(57) ABSTRACT

Blocking peptides comprised of the 14 amino acids that correspond to the sequences of the BB-loops of the four known TIR domain-containing adapter proteins (i.e. MyD88, TRAM, TIRAP, and TRIF) and homologous sequences of four TLRs (TLR2, TLR4, TLR1, and TLR6) are described. Adapter BB loop peptides disrupted TLR4, but not TLR2 signaling. TLR2 and TLR4 blocking peptides inhibited TLR4- and TLR2-mediated activation of ERK and cytokine induction, however, these peptides did not inhibit activation of p38. These peptides can be used to treat or prevent an immune or inflammatory response associated with a condition related to TLR4 signaling.

This application claims the benefit of priority from U.S. Provisional Application Ser. No. 60/672,133 filed on Apr. 15, 2005.

This invention was funded by the National Institutes of Health. The Government has certain rights in the invention pursuant to grant AI47233, AI57490, and HL69057.

### INTRODUCTION

Toll-like receptors (TLRs) are a family of signaling molecules that act as sensors for recognition of diverse pathogen-associated molecular patterns (PAMPs) or targets of innate immune recognition. All TLRs share a similar domain architecture: an extracellular region that contains multiple leucine-rich repeat regions (Rock et al. 1998, Proc. Natl. Acad. Sci. USA 95, 588) that function in recognition of PAMPs and co-receptors (Kobe and Kajava 2001, Curr. Opin. Struct. Biol. 11, 725); a transmembrane domain comprised of one membrane-spanning α-helix; and an intracellular “Toll-Interleukin-1 Resistance” (TIR) domain located within the C terminus (Rock et al., 1998, supra).

In addition to TLRs, a group of TIR domain-containing proteins that lack a transmembrane domain has been identified. Myeloid differentiation factor 88 (MyD88) was the first TIR domain-containing protein that was shown to serve as an adapter for IL-1, and later, for TLR signaling (Medzhitov et al., 1998, Mol. Cell. 2, 253). With rapid progress in sequencing and gene annotation, this family grew quickly and now includes five members, four of which are involved in TLR4 signaling: MyD88; TIR-domain containing adapter protein (TIRAP) (Horng et al. 2001, Nat. Immunol. 2, 825), also known as Mal (Fitzgerald et al., 2001, Nature 413, 78); TIR domain-containing adapter inducing IFN-β (TRIF) (Yamamoto et al., 2002, J. Immunol. 169, 6668), also referred as TIR domain-containing adapter molecule-1 (TICAM-1) (Oshiumi et al., 2003, Nat. Immunol. 4, 161); and TRIF-related adapter molecule (TRAM) (Fitzgerald et al., 2003, J. Exp. Med. 198, 1043), also named as TIR-containing protein (TIRP) (Bin et al., 2003, J. Biol. Chem. 278, 24526) or TICAM-2 (Oshiumi et al., 2003, J. Biol. Chem. 278, 49751).

MyD88 and TRIF have a more complex domain architecture than TIRAP and TRAM. In addition to the TIR domain, MyD88 has a mid-region and an N-terminal death domain that enable interaction with IL-1R-associated kinase 4 (IRAK-4) and IRAK-1, respectively, serine/threonine kinases that signal downstream of IL-1R (Muzio et al., Science 278, 1612) and TLRs (Medzhitov et al., 1998, supra), leading to NF-κB activation (reviewed in Janssens and Beyaert 2003, Mol. Cell. 11, 293). TRIF is the largest member of adapter family, with the TIR domain located in the middle of its sequence. The N-terminus of TRIF is necessary for activation of NF-κB and IRF-3 (Yamamoto et al., 2002, supra), a transcription factor responsible for induction of IFN-β and other genes. IKB kinase (IKK)-ε and TRAF family member-associated NF-κB binding kinase (TANK)-binding kinase-1 (TBK-1), act downstream of TRIF to phosphorylate IRF-3 (Fitzgerald et al., 2003, Nat. Immunol. 4, 491). Recruitment of IKK-ε and TBK1 to TRIF does not require TIR domain of the protein (Fitzgerald et al., 2003, J. Exp. Med. 198, 1043) and overexpression of a TRIF mutant that lacks the C-terminus still activates NF-κB and IFN-β reporter constructs (Yamamoto et al., 2002, supra; Oshiumi et al., 2003, supra).

TIRAP and TRAM are the smallest adapters involved in TLR signaling. The N-terminus of TRAM has a myristoylation site, the mutation of which alters its normal membrane localization (Vogel et al., 2003, Mol. Interv. 3, 12). Both TIRAP and TRAM have been found to associate constitutively with TLR4 (Fitzgerald et al., 2001, supra; Fitzgerald et al., 2003, J. Exp. Med. 198, 1043). Current models of TLR signaling ascribe different roles to the adapters (Oshiumi et al., 2003, supra; Akira and Takeda, 2004, Nat. Rev. Immunol. 4, 499). TIRAP and TRAM have been suggested to serve as “platform forming” components responsible for recruitment of the larger adapters, MyD88 and TRIF, respectively, that in turn, recruit downstream effector molecules (e.g., IRAK-1 and IRAK-4 to MyD88 and TBK-1, TRAF6, and IKK-ε to TRIF via non-TIR domains). Distinct combinations of adapters involved in a signaling platform differ among TLRs and have been postulated as the basis for the observed specificity of gene subsets induced by different TLR agonists (Vogel et al., 2003, supra; Akira and Takeda, 2004, supra, for review).

The structure of TLR TIR domains was first predicted by Rock et al. (1998, supra). Analysis of sequences of five huTLRs and their diverse homologs led the authors to conclude that TIR domains are composed of 5 β-strands alternating with 5 α-helices. Resolution of crystal structures of human TLR1 and TLR2 confirmed these theoretical findings and suggested that a functionally important proline-glycine combination that is highly conserved among TIR domains is located in the loop connecting the second β-strand with the second helix, the “BB-loop,” using the terminology proposed by Xu et al. (Xu et al., 2000, Nature 408, 111).

The importance of BB-loops in TLR signaling derives from observations that the function of TIR domain-containing proteins is highly susceptible to mutations in this region. A naturally occurring point mutation (P712H) in murine TLR4, which leads to complete unresponsiveness of the C3H/HeJ mouse to LPS (Poltorak et al., 1998, Science 282, 2085; Qureshi et al., 1999, J. Exp. Med. 189, 615), lies in this region. Similarly, replacement of Pro681 with His in human TLR2 was reported to disrupt signal transduction induced by Gram positive bacteria and essentially abolishes MyD88 recruitment (Xu et al., 2000, supra); however, this finding was not confirmed by Dunne et al. (Dunne et al., 2003, J. Biol. Chem. 2, 41443). Mutations of residues in the vicinity of the conserved BB-loop proline also led to the decreased activity of TLR2 (Xu et al., 2000, supra) and TLR4 (Ronni et al., 2003, Mol. Cell. Biol. 23, 2543). Many TIR-domain containing proteins that bear mutations homologous to the mutation in C3H/HeJ mice exert dominant-negative effects on TLR-signaling (e.g., TLR4 (Du et al., 1999, Blood Cells Mol. Dis. 25, 328), TLR2 (Underhill et al., 1999, Proc. Natl. Acad. Sci. USA 96, 14459), TIRAP (Horng et al., 2001 supra), and TRAM (Fitzgerald et al., 2003, J. Exp. Med. 198, 1043). Overexpression of the Pro200His mutation of MyD88, however, fails to prevent induction of NF-κB (Horng et al., 2001, supra) or its binding to human TLR4 (Dunne et al., 2003, supra). Similarly, in a yeast two hybrid system, interaction of TLR4 with TRAM is not disrupted by mutation of the TRAM cysteine 117 in its BB-loop (Dunne et al., 2003, supra).

Cell-penetrating cationic peptides (CPPs) are potent and efficient tools for delivery into the intracellular space of diverse substances that normally would not penetrate plasma membranes (Ford et al., 2001, Gene Ther. 8, 1). Cargoes that can be carried into cells vary widely in size and chemical nature and include oligonucleotides and proteins. After the initial observation that amino acids 47-57 of HIV-1 TAT protein are crucial for the ability of this protein to enter cells (Green and Loewenstein, 1988, Cell 55, 1179; Frankel and Pabo, 1988, Cell 55, 1189), functionally similar sequences were identified in the homeodomain of the Drosophila transcription factor, antennapedia (Joliot et al., 1991, Proc. Natl. Acad. Sci. USA 88, 1864), and the herpes-simplex-virus-1 DNA-binding protein, VP22 (HSV-1 VP22) (Eliott and O'Hare, 1997, Cell 88, 223). The mechanism by which CPPs enter cells is still controversial (Joliot and Prochiantz, 2004, Nat. Cell Biol. 6, 189).

For this study, we designed a set of “blocking peptides” (BPs) comprised of 14 aa sequence corresponding to the BB-loops of MyD88, TRAM, TIRAP, and TRIF. The translocating sequence of the antennapedia homeodomain was fused in tandem with these peptides. This approach was first used by Horng et al. (Horng et al, 2001, supra) in their original studies on the identification of TIRAP in TLR4 signaling. We tested these four constructs and a control peptide (CP) in primary murine macrophages for the ability to interfere with LPS-induced signal transduction. All four BPs blocked TLR4-mediated gene expression, MAPK and transacting factor activation, but failed to block TLR2-mediated activation of MAPKs. Only the MyD88 BP inhibited TLR2-mediated induction of IL-1β mRNA; however, its inhibitory effect was considerably weaker than its effect on TLR4-mediated IL-1β gene expression.

We took the same approach to design peptides which would structurally mimic the BB loop of the receptors TLR4, TLR2 and TLR 1 and TLR6. We found that TLR2 and TLR4 blocking peptides inhibited LPS-induced ERK phosphorylation nearly as efficiently as TRAM blocking peptide. Interestingly, the TLR2 and TLR4 BB loop blocking peptides also inhibit TLR2-mediated signaling, while TRAM BB loop selectively inhibited TLR4 only. Unexpectedly, in contrast to the adapter BB loop blocking peptides, the TLR2 and TLR4 BB loop peptides selectively inhibited ERK phosphorylation, but not p38 activation. In addition, the TLR1 and TLR6 BB loop blocking peptides did not inhibit ERK phosphorylation or gene expression, even though TLR2 heterodimerizes with TLR1 or TLR6 for signaling indicating that BB loops of TLR1 and TLR6 do not form interaction surfaces necessary for signaling from these receptors. Collectively, our findings suggest that these BPs interfere with protein-protein interactions involved in the assembly and/or stabilization of the TLR “signaling platform”, support the hypothesis that surfaces on MyD88 and TIRAP apart from the BB-loop enable their interaction with TLR2, and also suggest that different surfaces of TLR2 and TLR4 are utilized for assembly of signaling complexes that elicit activation of ERK and p38.

### SUMMARY OF THE INVENTION

We have found that adapter BB-loop peptides can selectively disrupt signaling pathways of TLR4 without significantly disrupting TLR2 signaling. As one of skill in the art understands, TLR4 is involved in numerous physiological responses, including infection, inflammation and cell damage in particular during graft rejection, and septic shock. Accordingly, the discovery of inhibitory peptides which can specifically inhibit or downregulate the activation and signaling pathway of TLR4 and block TLR4-mediated gene expression, MAPK and transacting factor activation is important for the development of effective treatments for conditions involving TLR4 signaling.

In addition, our data indicate that receptor blocking peptides differentially inhibit p-38 and ERK phosphorylation. These result allow fine tuning of treatments for conditions or diseases attributed to ERK phosphorylation and/or p38 phosphorylation.

Therefore, the aim of the present invention is to provide a composition and method for selectively modulating signaling of TLR4 and TLR4-mediated gene expression. Generally, this method includes introducing into a cell blocking peptides derived from BB-loops of adapter proteins, particularly TRAM, TRIF and MyD88, and to a lesser extent TIRAP, which interact with said TLR in an amount sufficient to produce said disruption.

It is another aim of the present invention to provide a composition and method for selectively inhibiting or modulating ERK phosphorylation and signaling, hence gene expression resulting from ERK phosphorylation without affecting p38 phosphorylation and resulting gene expression. Generally, this method includes introducing into a cell blocking peptides derived from receptor BB loops, e.g. TLR2 and/or TLR4, in an amount sufficient to produce said disruption.

Therefore, it is an object of the present invention to provide inhibitors of TLR4 signaling, both MyD88-independent and MyD88-dependent signaling pathways, by providing adapter blocking peptides comprising 14 amino acids derived from the BB-loop of the following adapter proteins:
(i) MyD88-BP, having the sequence Val Ser Asp Arg Asp Val Leu Pro Gly Thr Cys Val Trp Ser, (SEQ ID NO:1)
(ii) TRAM-BP, having the sequence Ile Val Phe Ala Glu Met Pro Cys Gly Arg Leu His Leu Gln, (SEQ ID NO:2)
(iii) TIRAP-BP, having the sequence Leu Gln Leu Arg Asp Ala Ala Pro Gly Gly Ala Ile Val Ser (SEQ ID NO:3)
(iv) TRIF-BP, having the sequence Phe Cys Glu Glu Phe Gln Val Pro Gly Arg Gly Glu Leu His (SEQ ID NO:4)

It is another object of the present invention to provide TRAM BB loop inhibitor peptides with amino acid substitutions as follows:
(i) P/H mutant, having the sequence Ile Val Phe Ala Glu Met His Cys Gly Arg Leu His Leu Gln (SEQ ID NO:5)
(ii) C/H mutant, having the sequence Ile Val Phe Ala Glu Met Pro His Gly Arg Leu His Leu Gln (SEQ ID NO:6)
(iii) E/A mutant, having the sequence Ile Val Phe Ala Ala Met Pro Cys Gly Arg Leu His Leu Gln (SEQ ID NO:7).

It is further another object of the present invention to provide a MyD88 blocking peptide based on the sequence of the “DD loop”, having the sequence Lys Tyr Lys Ala Met Lys Lys Asp Phe Pro Ser Ile Leu Arg (SEQ ID NO:8).

It is yet another object of the present invention to provide inhibitors of ERK phosphorylation and signaling, by providing receptor blocking peptides comprising 14 amino acids derived from the BB-loop of the following receptors:
(i) TLR-2, having the sequence Leu His Lys Arg Asp Phe Val Pro Gly Lys Trp Ile Ile Asp (SEQ ID NO:9)
(ii) TLR-4, having the sequence Leu His Tyr Arg Asp Phe Ile Pro Gly Val Ala Ile Ala Ala (SEQ ID NO:10).

It is another object of the present invention to provide a TLR1/6 blocking peptide based on the BB-loop sequence common to both TLR1 and TLR6, having the sequence Leu His Glu Arg Asn Phe Val Pro Gly Lys Ser Ile Val Glu (SEQ ID NO:11).

In certain embodiments, these blocking peptides can be linked to a cell-permeable peptide, for example the cell-permeable segment from the antennapedia homeodomain or from HSV-1 VP22. In another aspect, the present invention provides these peptides in pharmaceutically acceptable forms and in pharmaceutical compositions including these peptides, pharmaceutically acceptable forms of the peptides, derivatives thereof, or pro-drugs thereof.

In yet another aspect, the present invention provides methods for using the blocking peptides for treating or ameliorating a condition treatable by selectively inhibiting signaling of TLR4 and TLR4-mediated gene expression in a subject, and/or treating or ameliorating a condition treatable by selectively inhibiting TLR-induced ERK phosphorylation and ERK-mediated gene expression. Conditions include treatment of inflammation and affecting immune response or reducing physiological conditions resulting from excess TLR4 activation including infection and cell damage, and treatment of conditions induced by excessive activation of this branch of TLR signaling. Generally, the method includes administering to the subject an amount of the blocking peptides or a pharmaceutically acceptable form of one or more of the blocking peptides effective for treating or ameliorating the condition.

Various other features and advantages of the present invention should become readily apparent with reference to the following detailed description, examples, claims and appended drawings. In several places throughout the specification, guidance is provided through lists of examples. In each instance, the recited list serves only as a representative group and should not be interpreted as an exclusive list.

### BRIEF DESCRIPTION OF THE DRAWINGS

FIG. 1. Effect of blocking peptides on the cytokine induction by LPS. Thioglycollate-elicited primary murine macrophages were preincubated in the presence of 40 μMof BP for one hour before stimulation with 10 ng/ml of LPS. (A) Quantitative real-time PCR analysis of gene expression. (B) Secretion of IL-1β and IL-6. Concentration of the cytokines was measured after overnight incubation of the cells in the presence of LPS and the peptides. (C) Sensitivity of the LPS-stimulated expression of IFN-β and IL-1β mRNA to varying concentrations of TRAM and MyD88 BP. Total mRNA was isolated 1 hour after LPS stimulation (A and C). *p<0.05; **p<0.01.

FIG. 2. Effect of blocking peptides on the LPS-stimulated activation of MAPKs and Tyr701 STAT-1. Ten μg of total protein was loaded per lane. A representative blot of three separate experiments is shown. Other details are given in the Materials and Methods section and legend for FIG. 1.

FIG. 3. Effect of blocking peptides on activation of IRF-3 and IRF-1. The upper panel shows native PAGE blot, the two below represent the same lysates run on SDS-PAGE. Primary mouse macrophages were stimulated with 100 ng/ml of LPS. Seventeen μg of total protein was loaded per lane of the native gel. Other details are as described in the legend for FIG. 1.

FIG. 4. Comparative effect of BPs on TLR2-vs. TLR4-mediated activation of MAPKs (A) and IL-1β mRNA expression (B). For Western blot analysis, 4.5 x10⁶ primary macrophages were preincubated without or with 40 μM BP and stimulated for 30 min with LPS (10 ng/ml) or Pam3Cys (P3C; 150 ng/ml). The results shown are a representative blot of three separate experiments. Experimental details for FIG. 4B are the same as reported in the legend to FIG. 1A.

FIG. 5. Effect of mutated TRAM BB loop peptides on LPS-induced activation of ERK. Details of the experiment are as in legend for FIG. 4, except for the cells were preincubated with peptides only for 30 minutes.

FIG. 6. Effect of mutant TRAM BB loop peptides on LPS-induced activation of IL1-β and IFN-β mRNA. Details of the experiments are as described in the legend for FIG. 1, except for the cells were preincubated with peptides only for 30 minutes.

FIG. 7. Antennapedia DD loop peptide does not inhibit LPS- or P3C-induced activation of ERK. Details of experiments are as in legend for FIG. 5.

FIG. 8. TLR2 and TLR4 BB peptides inhibit LPS-, and to some extent, P3C-induced activation of ERK. For details of experiments, see legend for FIG. 4.

FIG. 9. BB loop peptides inhibit LPS-induced activation of ERK in a non-competitive manner. For details of experiments, see legend for FIG. 4.

FIG. 10. Selective effect of TLR2 and TLR4 BB peptides on activation ERK and p38. For details of experiments, see legend for FIG. 4.

FIG. 11. Effect of TLR2 and TLR4 BB peptides on induction of IL1-β and IFN-β mRNA by LPS. For details of experiments, see legend for FIG. 6.

FIG. 12. Comparative effect of TLR1/6 BB peptide on LPS- and P3C-induced activation of MAP kinases and induction of IL1-β and IFN-β mRNA.

### DETAILED DESCRIPTION

The following definitions are provided to facilitate understanding of certain terms used frequently herein.

“Peptide” refers to any peptide or protein comprising two or more amino acids joined to each other by peptide bonds or modified peptide bonds, i.e. peptide isosteres. “Peptide” refers to both short chains, commonly referred to as peptides, oligopeptides or oligomers, and to longer chains, generally referred to as proteins. Peptides may contain amino acids other than the 20 gene-encoded amino acids. Peptides include amino acid sequences modified either by natural processes, such as post-translational processing, or by chemical modification techniques which are well known in the art. Such modifications are well described in basic texts and in more detailed monographs, as well as in research literature. Modifications may occur anywhere in a peptide, including the peptide backbone, the amino acid side-chains and the amino or carboxyl termini. It will be appreciated that the same type of modification may be present to the same or varying degrees at several sites in a given peptide. Also, a given peptide may contain many types of modifications. Peptides may be branched as a result of ubiquitination, and they may be cyclic, with or without branching. Cyclic, branched and branched cyclic polypeptides may result from post-translation natural processes or may be made by synthetic methods. Modifications include acetylation, acylation, ADP-ribosylation, amidation, covalent attachment of lavin, covalent attachment of a lipid or lipid derivative, covalent attachment of phosphatidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation proteolytic processing, phosphorylation, prenylation, recemization, slenoylation, sulfation, transfer-RNA mediated addition of amino acids to proteins such as arginylation, and ubiquitination (see, for instance, PROTEINS—STRUCTURE AND MOLECULAR PROPERTIES, 2^{nd} Ed., T. E. Creighton, W.H. Freeman and Co., New York, 1993; Wold, F., Post-translational Protein Modifications: perspectives and Prospects, pgs. 1-12 in POSTRANSLATIONAL COVALENT MODIFICATION OF PROTEINS, B. C. Johnson, Ed., Academic Press, New York, 1983; Seifter et al., Analysis for protein modification and nonprotein cofactors, Meth. Enzymol., 1990, 182: 626-646 and Rattan et al., Protein Synthesis: Post-translational Modifications and Aging, Ann NY Acad Sci, 1992, 663, 48-62.)

In a first aspect, the present invention relates to blocking peptides derived from the BB-loops of TLR4 adapter proteins. The adapter BB-loops described herein are from known TLR4 adapter proteins. Other yet unknown TLR4 adapter proteins containing a BB-loop are intended to be part of this invention and would be expected to function similarly to the herein exemplified BB-loop peptides. Specifically, the polypeptides set forth in SEQ ID NO:1, comprising the MyD88 BB-loop sequence, SEQ ID NO:2, comprising TRAM BB-loop sequence, SEQ ID NO:3, comprising TIRAP BB-loop sequence, SEQ ID NO:4, comprising TRIF BB-loop sequence. Additionally, adapter peptides having one or more mutations or deletions such that peptide function or binding affinity to the receptor are retained or altered are part of this invention. For example, the peptides specified in SEQ ID NO:5, comprising TRAM BB-loop mutant P/H, SEQ ID NO:6, comprising TRAM BB-loop mutant C/H, SEQ ID NO:7, comprising TRAM BB-loop E/A mutant.

These peptides exhibit an inhibitory activity on TLR4 downstream signaling but not on TLR2 signaling. The term “block” and variations thereof refer to any measurable decrease in expression of products of TLR4 activation, signaling, or TLR4-mediated gene expression, for example, cytokine gene expression. Preferred blocking peptides of the present invention having homology to any of the peptides identified in SEQ ID NO:1-7, as explained further below are expected to have similar functions/properties to these peptides. These peptides can be used individually or in any combination. Our results indicate that TRAM-BP is the most potent inhibitor, with MyD88-BP and TRIF-BP being the next most potent inhibitors. Providing all the peptides together may produce a synergy of action.

These preferred peptides block both the MyD88-independent and the MyD88-dependent response of TLR4 activation and interfere with the induction of immediate response genes such as ERK, JNK, phosphorylation of MAPK, dimerization of interferon regulatory factor 3 (IRF3), IRF-1, IL-1β, IL-6, MIP1B, IFN-β, and RANTES producing a reduction in inflammation and the inflammatory response.

In a second aspect, the present invention relates to blocking peptides derived from the BB-loops of TLR receptors, specifically, TLR2 BB-loop peptide specified in SEQ ID NO:9, TLR4 BB-loop peptide specified in SEQ ID NO:10, and TLR1/6 BB-loop peptide specified in SEQ ID NO:11. TLR peptides of the present invention having homology to any of the peptides identified in SEQ ID NO:9-11 are included as part of the invention. All TLRs have a BB loop, in some cases there are slight interspecies differences in sequence, e.g. TIR domains from human MyD88, TLR4 and all four TLR1/6 are identical to the mouse sequences, while others have one or two synonymous replacements. Peptides derived from other TLR receptors of the same or different species, having BB-loops are considered part of this invention and would be expected to function in a similar manner, i.e. block a factor or peptide from binding to the TLR from which the blocking peptide is derived, or compete with the TLR for binding to a factor which recognizes the region from which the blocking peptide is derived. The term “block” and variations thereof refer to any measurable decrease in binding of a factor to TLR, TLR activation, signaling, or TLR-mediated gene expression.

Our results indicate that 1) receptor and adapter blocking peptides sequester different pools of signaling molecules; while adapter BB peptides show strong selectivity towards blocking TLR4 signaling and inhibit downstream gene expression, receptor BB peptides are not selective for specific branches of TLR signaling, i.e. they were not able to inhibit p38 activation. 2) TLR2 and TLR4 peptides show cross-reactivity, i.e. TLR2 BB peptides can inhibit both TLR2- and TLR4-induced signaling, similarly for TLR4 BB peptide. 3) BB loops of TLR1 and TLR6 do not form important interactive surfaces necessary to signaling from these receptors. 4) Despite limited inhibitory activity of receptor peptides towards activation of p38, these constructs effectively block induction of cytokines by TLR2 and TLR4 agonists, the leading cause for propagation of inflammation. Therefore, by choosing an appropriate receptor blocking peptide or peptides, selective inhibition of signaling from one or more targeted TLR(s) can be achieved.

In a particular embodiment of the invention, the present invention relates to a blocking peptide whether derived from a BB loop of a TIR domain containing protein which includes, but is not limited to, TLR adapter proteins, TLR receptor proteins, and other proteins which are not involved in TLR signaling but use the same mechanism of signaling as TLR's, e.g. IL1-type receptors can be linked or fused to a cellular membrane transport protein. By “linked” as used herein is meant that the biologically active molecule is associated with the cell-permeable peptide in such a manner that when the cell-permeable peptide crosses the cell membrane, the molecule is also imported across the cell membrane. Examples of such means of linking include a peptide bond, i.e. the two peptides can be synthesized contiguously. Alternatively, a non-peptide covalent bond can be used, such as conjugating a cell-permeable peptide to a protein with a cross-linking reagent, for example, glutaraldehyde. The molecules can be simply mixed with the cell-permeable peptide and thus allowed to associate. Additionally, standard chemical ligation methods, such as using chemical cross-linkers interacting with the carboxy-terminal amino acid of the cell permeable peptide can be utilized. All these methods are standard in the art.

Therefore, in one aspect, the present invention comprises one or more fusion protein comprising the blocking peptides described above, or an active portion or active fragments thereof, and a cellular membrane transport protein. By fusion protein, as disclosed herein, means any protein that includes a membrane permeable sequence as disclosed herein, attached or linked to any blocking peptide as disclosed herein. It is also understood that the fusion protein of the present invention can include multimers (e.g. heterodimers or homodimers) or complexes with itself or other proteins. As a result, pharmaceutical compositions as described herein may comprise a protein of the invention in such multimeric or complexed forms. The membrane-permeable sequence as disclosed herein may be located immediately adjacent to, or some distance from, the blocking peptide as disclosed herein. Therefore, it is also understood that by “fusion protein” is also intended to include any peptide or cell-permeable sequence either N-terminal or C-terminal to the blocking sequence, or both.

Any cell-permeable peptide capable of translocating across the cell membrane into the interior of a cell can be used according to this invention. Several methods of entry into the cells are known such as endocytosis, direct transport across the lipid bilayer, and receptor-mediated entry. Viruses or virus fragments, such as HSV-1

VP22 can be used to transport proteins into cells, in addition to the cell-permeable segment of the Antennapedia homeodomain, obtained from the fruit fly Drosophila having sequence Arg Gln Ile Lys Ile Trp Phe Gln Asn Arg Arg Met Lys Trp Lys Lys (SEQ ID NO:12). Functional examples of a fusion protein of the instant invention, and particularly a fusion of cell-permeable segment of Antennapedia homeodomain with the blocking peptides described above are identified as SEQ ID NO:13-17 (see Table I below).

Alternatively, the blocking peptides are delivered into the cells by other means such as physical methods of introducing proteins into cells e.g. microinjection, electroporation, biolistics, chemical or biological pore formation, e.g. digitonin, pore forming proteins and ATP treatment, use of modified proteins, lapidated proteins and bioconjugates, such as with an immunotoxin, and particle uptake, microspheres, virus mimics, induced pinocytosis.

As one of skill in the art understands, an organic compound, typically with a molecular weight under 2 KD, having appropriate structural similarity to the blocking peptides described above may also be used as blockers of TLR4 activation. For example, a small molecule mimetic may be used. Small molecule mimetics as known in the art, are chemically synthesized compounds that provide the spatial conformation necessary to properly associate to a particular protein and elicit a response. Therefore, the present invention also comprises the use of small molecule mimetics to inhibit TLR signaling.

TABLE I
The physical-chemical properties of BPs^{a}

| | | | | | | | |
|---|---|---|---|---|---|---|---|
| | | | | # of | # of | | |
| | | | | positively | negatively | | Hydro- |
| Peptide, | Sequence, | (SEQ ID NO:), | Length, | charged aa, | charged aa, | AI^{b,} | pathicity |

| | | | | | | | |
|---|---|---|---|---|---|---|---|
| MyD88-BP | RQIKIWFQNRRMKWKKVSDRDVLPGTCVWS | (SEQ ID NO: 13) | 30 | 8 | 2 | 68.00 | −0.850 |
| mu/hu | | | | | | | |
| TRAM-BP | RQIKIWFQNRRMKWKKIVFAEMPCGRLHLQ | (SEQ ID NO: 14) | 30 | 8 | 1 | 78.00 | −0.637 |
| mu | | | | | | | |
| TIRAP-BP | RQIKIWFQNRRMKWKKLQLRDAAPGGAIVS | (SEQ ID NO: 15) | 30 | 8 | 1 | 71.67 | −0.723 |
| mu | | | | | | | |
| TRIF-BP | RQIKIWFONRRMKWKKFCEEFQVPGRGELH | (SEQ ID NO: 16) | 30 | 8 | 3 | 48.67 | −1.190 |
| mu/hu | | | | | | | |
| TLR1/6-BP | RQIKIWFQNRRMKWKKLHERNFVPGKSIVE | (SEQ ID NO: 17) | 30 | 9 | 2 | 71.33 | −1.103 |
| mu/hu | | | | | | | |
| TLR2-BP | RQIKIWFQNRRMKWKKLHKRDFVPGKWIID | (SEQ ID NO: 18) | 30 | 10 | 2 | 74.67 | −1.110 |
| mu | | | | | | | |
| TLR4-BP | RQIKIWFQNRRMKWKKLHYRDFIPGVAIAA | (SEQ ID NO: 19) | 30 | 8 | 1 | 84.67 | −0.567 |
| mu/hu | | | | | | | |
| Control | RQIKIWFQNRRMKWKKSLHGRGDPMEAFII | (SEQ ID NO: 20) | 30 | 8 | 2 | 68.33 | −0.877 |
| peptide | | | | | | | |

^{a}The physical-chemical properties of peptides were calculated using ExPASY-ProtParam Tool (Swiss Institute of Bioinformatics, Geneva).
^{b}AI-peptide aliphatic index, relative volume occupied by aliphatic chains of A, V, I, L.

The blocking peptides of the present invention can be prepared by methods known in the art, for example chemical synthesis, in linear order from the amino-terminal end, optionally a cell-permeable peptide sequence, an optional spacer amino acid region, and a blocking peptide described above. Such a peptide could also be produced through recombinant DNA techniques, expressed from a recombinant construct encoding the above-described.

In a further aspect, the present invention provides methods of treating conditions related to excess activation of TLR4. One method comprises administering to a subject in need one or more blocking peptide or mimetic as described above, optionally in combination with a pharmaceutically acceptable carrier, in an amount effective to inhibit or reduce TLR4MyD88-dependent or MyD88-independent signaling.

In another aspect, the present invention provides methods for treating conditions related to excess ERK phosphorylation or signaling and/or treating or ameliorating a condition treatable by selectively inhibiting ERK phosphorylation and ERK-mediated gene expression. One method comprises administering to a subject in need of such treatment one or more receptor blocking peptide, preferably TLR4 blocking peptide and/or TLR2 blocking peptide, in an amount effective to inhibit or reduce ERK phosphorylation and/or ERK signaling.

Any selected cell into which import of a biologically active molecule of the present invention would be useful can be targeted, as long as there is means to bring the complex in contact with the selected cell. Cells can be within a tissue or organ, for example, supplied by a blood vessel into which the complex is administered. Additionally, the cell can be targeted by, for example, inhalation of the molecule linked to the peptide to target the lung epithelium. Some examples of cells that can be targeted include epithelial cells, endothelial cells, blood cells and tumor cells, among many. In addition, the complex can be administered directly to a tissue site in the body. As discussed above, the cell-permeable peptide utilized can be chosen from peptides known to be utilized by the selected target cell, or can be tested for importing ability given the teachings herein. Generally, however, all cell-permeable peptides have the common ability to cross cell membranes due, at least in part, to their hydrophobic character. Thus, in general, a membrane-permeable peptide can be designed and used for any cell type, since all eukaryotic cell membranes have a similar lipid bilayer.

Administration of the blocking peptides may be in vitro or in vivo. In vitro application may include collecting from a subject, a sample of immune cells, endothelial cells, mucosal epithelium of the lung or gut, or any other kind of cell in which treatment is beneficial, culturing the collected cells in vitro, and adding the selected blocking peptide or peptides to the cell culture. After the cells have been exposed to the blocking peptide or peptides, the treated cells may be reintroduced into the subject, thereby providing therapeutic or prophylactic treatment. In vivo application may include administering the selected blocking peptide or peptides to a subject. The selected peptide or peptides may be administered in any suitable manner including but not limited to topical by salves, pastes, gels and the like, injection (e.g. intravenous, subcutaneous, intraperitoneal, intradermal), inhalation, ingestion, transdermal using penetrants such as bile salts or fusidic acids or other detergents, or transmucosal delivery. If the peptides of the invention can be formulated in an enteric or an ecapsulated formulation, oral administration may also be possible. In addition, the compounds of the invention may be administered in combination with another compound or active agent including immunogens, adjuvants, antivirals, antibiotics, anti-inflammatory agents, etc, such as aspirin, diflunisal, mesalamine, salicylsalicylic acid, sodium thiosalicylate, choline salicylate, magnesium salicylate, olsalazine, sufasalazine, indomethacin, suldinac, etodolac, mefenamate, meclofenamate, flufenamate, tolfenamate, etofenamate, tolmetin, ketorolac, diclofenac, ibuprofen, naproxen, fenoprofen, ketoprofen, flurbiprofen, oxaprozin, piroxicam, meloxicam, nabumetone, apazone, nimesulide, zileuton, gold salts, colchicines, allopurinol, beclomethasone, budesonide, flunisolide, triamcinolone, prednisone, cromolyn, nedocromil, albuterol, bitolterol, pirbuterol, salmeterol, terbutaline, theophylline, and other methylxanthines, metaproterenol, systemic glucocoticoids, antiprotozoal agents, antimalarial agents, isoniazid rifampin, ethambutol, antifungal agents, alkylating agents, and antimetabolites, retinal tretinoin, isotretinoin, etretinate, acitretin, arotinoid, beta-carotene, calcipotriene, anthralin, psoralen, 5-methoxypsoralent, trioxsalen, coal tar, masoprocol and any pharmaceutically acceptable prodrug or derivative thereof.

In a further aspect, the present invention provides for pharmaceutical compositions comprising a therapeutically effective amount of one or more blocking peptide, such as the soluble form of the present invention, or small molecule compound, in combination with a pharmaceutically acceptable carrier or excipient. Such carriers include, but are not limited to, saline, buffered saline, dextrose, water, glycerol, ethanol, and combinations thereof. The invention further relates to pharmaceutical packs and kits comprising one or more containers filled with one or more of the ingredients of the aforementioned compositions of the invention.

The dosage range required depends on the choice of blocking peptide or other compounds (such as small molecule mimetic) of the present invention, the route of administration, the nature of the formulation, the nature of the subject's condition, and the judgment of the attending practitioner. Suitable dosage, however, are in the range of 0.1-100 ug/kg of subject. Wide variations in the needed dosage are to be expected in view of the variety of compounds available and the differing efficiencies of various routes of administration. For example, oral administration would be expected to require higher dosages than administration by intravenous injection. Variations in these dosage levels can be adjusted using standard empirical routines for optimization, as is well understood in the art.

Peptides used in treatment can also be generated endogenously in the subject, referring to “gene therapy”. Thus, for example, cells from a subject may be engineered with a polynucleotide, such as DNA or RNA, to encode a polypeptide ex vivo, and for example, by the use of retroviral plasmid vector. The cells are then introduced into the subject.

Treating a condition may involve either prophylactic or therapeutic treatment. As used herein, proplylactic treatment refers to treatment initiated before the onset of symptoms or signs of the condition. Thus, prophylactic treatments generally are designed to: (i) reduce the likelihood that the subject receiving the treatment will acquire the condition, (2) reduce the severity of the condition, once acquired, or (3) both. As used herein, therapeutic treatment refers to treatment initiated after the onset of symptoms or signs of a condition. Thus, therapeutic treatments are designed to limit or reduce progression of the condition. In some cases, therapeutic treatments can result in reversal of the condition, even to the point of complete resolution.

Conditions that may be treated using method of the present invention include, but are not limited to viral diseases, bacterial diseases, fungal diseases, parasitic diseases, neoplastic diseases, neurodegenerative diseases, stroke, cardiovascular diseases, TH2-mediated atopic diseases, autoimmune diseases, and diseases associated with wound repair.

In one embodiment, the present invention provides a method for treating or preventing sepsis (septic shock) in a subject, e.g. a human subject, comprising delivering to the subject a compound comprising the blocking peptides of the present invention such that TLR4 signaling is inhibited or reduced. Alternatively, a nucleic acid encoding one or more blocking peptides could be delivered for example as naked DNA, with a viral vector, or by means such as cationic liposomes. The peptide used in this and any other method of the invention can further include a cell-permeable region.

Furthermore, the present method inhibits the TLR4-mediated gene expression and production or activation of factors resulting from TLR4 activation, such as STAT-1, interferon receptor activation, IFNβ, RANTES, IL-1, IL-6, TNFα, ERK, and JNK.

The methods and peptides of the invention can be used to treat or prevent inflammatory responses caused by a microbe (or a toxin from a microbe), e.g. a bacterium (e.g. a Gram-positive or Gram-negative bacterium), such as E. coli, Salmonella typhimurium, Salmonella typhosa and other Salmonella species, or Pseudomonas aeruginosa and other Pseudomonas species; or the bacterium can be a Gram-positive bacterium, such as a species of Staphylococcus, Streptococcus, and Pneumococcus that causes an inflammatory response, for example, as a result of food poisoning or a noscomial infection). Other example of microbial infections that cause inflammatory responses that can be treated or prevented by the methods of the invention include rickettsia, e.g. Rickettsia rickettsiae; a virus, e.g. Ebola virus, Dengue hemorrhagic fever virus, West Nile encephalitis virus, and hepatitis virus A, B, or C; fungi, e.g. Candida albicans, Cryptococcus neoformans, and Histoplasma capsulatum), and protozoans, e.g. Plasmodium falciparum and other species of Plasmodium that cause malaria.

The methods and peptides of the invention can be used to treat or prevent inflammatory reactions triggered by toxins, such as any toxin produced by a microbe that causes an inflammatory response, for example, but not limited to, lipoplysaccharide, or a superantigen (e.g. Staphylococcus enterotoxin A or B, streptococcal pyrogenic toxins and M proteins, or any superantigen produced by a microbe). The methods can also be used to treat or prevent any inflammatory reactions that can be treated by the methods of the invention including plant toxins, e.g. poison ivy or poison oak, nickel, latex, environmental toxins (such as toxic chemicals) or allergens that invoke an inflammatory response upon skin contact or inhalation. For example, inhalation of toxins can cause adult respiratory distress syndrome (which can also result from septic shock and other medical conditions), which can be treated or prevented using the methods of the invention.

Both systemic and localized inflammatory responses can be treated or prevented using the methods and peptides of the invention. The methods and peptides of the invention can also be used to treat or prevent inflammatory responses that affect the function of specific organs or organ systems, for example, but not limited to, the liver, bowel, kidney, joints, skin, pancreas, central nervous system, peripheral nervous system, bladder, or reproductive organs. In some cases, the inflammatory response is caused by an inflammatory disease, for example, an autoimmune disease. Examples of such autoimmune diseases include, but are not limited to, inflammatory bowel disease, Crohn's disease, glomerulonephritis, multiple sclerosis, lupus erythematosis, rheumatoid arthritis, psoriasis, or juvenile diabetes. The methods and peptides of the invention can also be used to treat chronic or acute inflammatory diseases and conditions of the skin, for example, psoriasis, eczema, or contact dermatitis.

Moreover, cellular apoptosis induced by inflammatory conditions involving pro-inflammatory cytokines and/or nuclear import of stress-responsive transcription factors (such as NFκB or STAT-1) can be inhibited, minimized, or prevented using the methods of the invention. For example, apoptosis of liver cells resulting from septic shock can be inhibited by the present methods and peptides. These can be used to used to inhibit liver cell apoptosis caused by other types of acute liver injury resulting from inflammation, for example, toxins that poison the liver (one example being poisoning by acetomenaphen) or viruses (such as hepatitis virus. The present invention also provides kits which are useful for carrying out the present invention. The present kits comprise a first container means containing one or more of the above-described peptides. The kit also comprises other container means containing solutions necessary or convenient for carrying out the invention. The container means can be made of glass, plastic or foil and can be a vial, bottle, pouch, tube, bag, etc. The kit may also contain written information, such as procedures for carrying out the present invention or analytical information, such as the amount of reagent contained in the first container means. The container means may be in another container means, e.g. a box or a bag, along with the written information.

All publications, including, but not limited to patents and patent applications, cited in this specification are herein incorporated by reference as if each individual publication were specifically and individually indicated to be incorporated by reference herein as though fully set forth.

The invention is further described in detail to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided therein.

### Materials and Methods

Animals

C3H/OuJ mice were purchased from the Jackson Laboratory (Bar Harbor, Me.). Thioglycollate-elicited peritoneal macrophages were harvested and cultured in the presence of E. coli K235 LPS or Pam3Cys as described previously (Salkowski et al., 1999, J. Immunol. 164, 1529).

Design of Peptides

The peptides were composed of 16 aa carrier sequence from Drosophila homeodomain protein (amino acids 366-381 (SEQ ID NO:12), GI: 4389425), the C-terminus of which was synthesized in tandem with the N-terminus of the 14 aa that surround the conserved P(C)G sequence in BB-loops of murine TIR domain-containing adapter proteins: TRAM (SEQ ID NO:2) (GI: 27734184), MyD88 (SEQ ID NO:1) (GI: 6754772), TRIF (SEQ ID NO:4) (GI: 33859797) and TIRAP (SEQ ID NO:3) (GI: 16905131) (sequences for fused peptides provided in Table I). A control peptide (CP) was a scrambled amino acid sequence shown by BLAST to not be homologous to known proteins (see Table I, the last 14 aa of SEQ ID NO:20). Peptides were synthesized and purified by HPLC by Biosynthesis, Inc. (Lewisville, Tex.). Purity was confirmed by mass spectrometry. Ten mM stocks were dissolved in 25% DMSO and kept frozen at −80° C. Forty μM was the highest concentration used in these experiments due to limited solubility of BPs in the cell culture medium.

Isolation of mRNA and RT-PCR

Total cellular RNA was extracted from macrophage cultures as described elsewhere and reversed transcribed using AMV Reverse Transcriptase (Promega, Madison, Wis.) and poly-T priming as recommended by the manufacturer. The resulting cDNA was quantified by real time PCR using SYBR Green PCR Master Mix (Applied Biosystems) and ABI Prism 7900HT cycler. Primers for detection of IL-1β, IFN-β, MIP-1β, □RANTES, and HPRT mRNAs were designed using the Primer Express 2.0 program (Applied Biosystems). Relative gene expression was calculated using ΔCt method with HPRT as a housekeeping gene (1.8̂(Ct_{HPRT}−Ct_{Gene})) Statistical analysis was performed by one way ANOVA with repeated measures, followed by Dunnett's multiple comparison post hoc test with p≦0.05 chosen as the level of significance.

SDS-PAGE and Western Analysis

SDS-PAGE and Western blot analysis were performed as described previously (Medvedev et al., 2001, J. Immunol. 167, 2257) with minor modifications. Antibodies for detection of activated MAPKs (pERK, pJNK and pP38) were purchased from Cell Signaling Technology (Beverly, Mass.). IRF-1 was detected using a rabbit polyclonal antibody from Santa-Cruz (Santa Cruz, Calif.). Antibody for detection of tyrosine-701 phosphorylated STAT1 was purchased from Zymed Laboratories (San Francisco, Calif.).

Native PAGE for detection of IRF-3 activation was performed with minor modifications (Iwamura et al., 2001, Genes Cells 6, 375). One×10⁷ thioglycollate-elicited cells were plated into a 6 cm dish and washed with PBS to remove non-adherent cells. Primary macrophages were stimulated with 100 ng/ml of LPS for 1.5 hr and lysed in buffer containing 50 mM TRIS-HCl pH 7.5, 150 mM NaCl, 1 mM EDTA, 1% NP-40, 1 mM sodium orthovanadate and 1× Complete protease inhibitor cocktail (Roche, Mannheim, Germany). Lysates were mixed with native lysis buffer (Bio-Rad, Hercules, Calif.) supplemented with 1% deoxycholate. Fifteen to 20 μg protein per lane was electrophoresed on a non-reducing gel and immunoblotted. IRF-3 dimers and monomers were detected using rabbit anti-IRF-3 antibody (Zymed Laboratories) by Western analysis.

### Example 1

BB-Loop Peptides Block LPS-Induced Gene Expression in Primary Macrophages, but do not Distinguish Between MyD88-Dependent and Independent Pathways

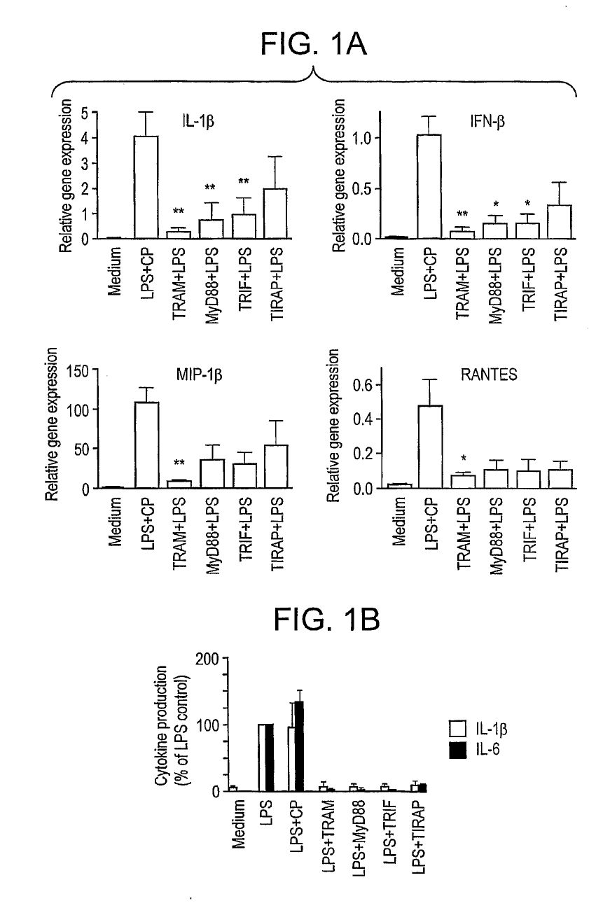

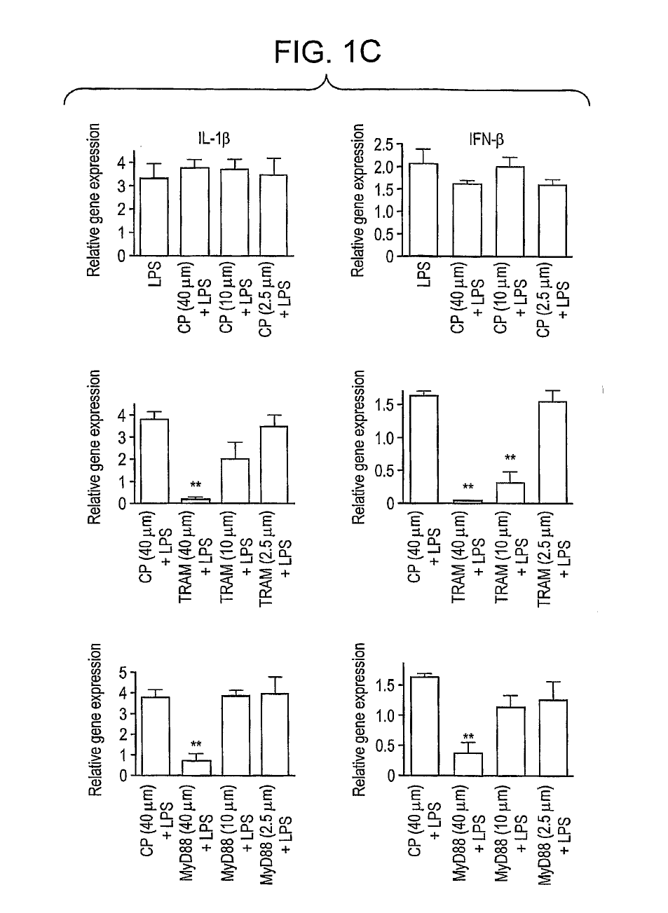

Based on the observation that macrophages from MyD88 knockout mice do not synthesize TNF-α or IL-6 in response to LPS, while are still capable of inducing IFN-β and IFN-β-inducible genes (Kawai et al., 2001, J. Immunol. 167, 5587; Toshchakov et al., 2002, Nat. Immunol. 3, 392), two different signaling pathways propagating from TLR4 were defined (reviewed in Akira and Takeda, 2004, supra). The “MyD88-independent” pathway of TLR4 signaling utilizes TRAM and TRIF to activate IRF-3 that, in turn, leads to induction of IFN-β and IFN-β-inducible genes, while the “MyD88-dependent” pathway leads to induction of genes such as IL-1β, TNF-α, and IL-6 that do not depend on IRF-3 for their expression. Mice with targeted mutations in MyD88 (Kawai et al., 2001, supra; Toshchakov et al., 2002, supra) or TIRAP (Yamamoto et al., 2002, Nature 420, 324) retain the capacity to signal through the MyD88-independent pathway. In contrast, the effect of targeted mutations in TRIF or TRAM affects both arms of the TLR4 signaling pathway: macrophages derived from TRIF and TRAM knockouts not only exhibited a complete loss of “MyD88-independent” signaling, but also exhibited a severe loss of signaling associated with the MyD88-dependent pathway (e.g., TNF-α, IL-6 and IL-12 p40 secretion is essentially eliminated, although there is some retention of early NF-κB translocation) (Yamamoto et al., 2003, Nat. Immunol. 4, 1144; Yamamoto et al., 2003a, Science 301, 649). Therefore, the first question we sought to address experimentally is whether interactions of the BB-loops of the TIR domains of these adapters affect protein interactions leading to gene expression. To this end, we designed cell permeable peptides whose sequences correspond to the BB-loops of each adapter molecule and measured the ability of each BP to interfere with the induction of immediate response genes induced via MyD88-dependent and independent pathways. FIG. 1A shows the effect of the BP treatment of macrophage cultures on the induction of two MyD88-dependent genes, IL-1β and MIP-1β(Bjorkbacka et al., 2004, Physiol. Genomics 19, 319; Kawai et al., 1999, Immunity 11, 115), and two MyD88-independent genes, IFN-β and RANTES (Kawai et al., 2001, supra; Toshchakov et al., 2002, supra; Bjorkbacka et al., 2004, supra), 1 h after LPS stimulation of primary macrophage cultures. All BB-loop-containing peptides exerted an inhibitory effect on LPS-induced expression of MyD88-dependent IL-1β and MIP-1⊕, as well as MyD88-independent IFN-β and RANTES mRNA, as measured by real-time PCR (FIG. 1A). In general, the inhibitory effect of the TRAM BP was strongest, with MyD88 BP being comparable or only slightly less inhibitory; the TIRAP BP was consistently weakest among the four tested. FIG. 1B demonstrates the effect of BB-loop peptides on IL-1β and IL-6 protein expression. The BPs, but not the CP, also blocked secretion of IL-1β and IL-6. The concentration of BP used in FIG. 1 and all other experiments was 40 μM based on optimal inhibitory activity of the two most active BPs, TRAM BP and MyD88 BP, against an MyD88-dependent gene (e.g., IL-1β) and an MyD88-independent gene (e.g., IFN-β) (FIG. 1C). FIG. 1C also shows that BP-mediated inhibition is seen over a very narrow concentration range (i.e., between 10 and 40 μM).

### Example 2

### Blocking Peptides Inhibit Activation of MAPKs by LPS

Since LPS-induced gene expression and cytokine secretion are consequences of signaling pathways induced by both the MyD88-dependent and independent signaling pathways, the data presented in FIG. 1 support the hypothesis that the BPs sequester and/or block target proteins of the adapter BB-loops that are essential for both arms of the TLR signaling pathway. Therefore, we next sought to examine upstream signaling pathways for their sensitivity to BPs.

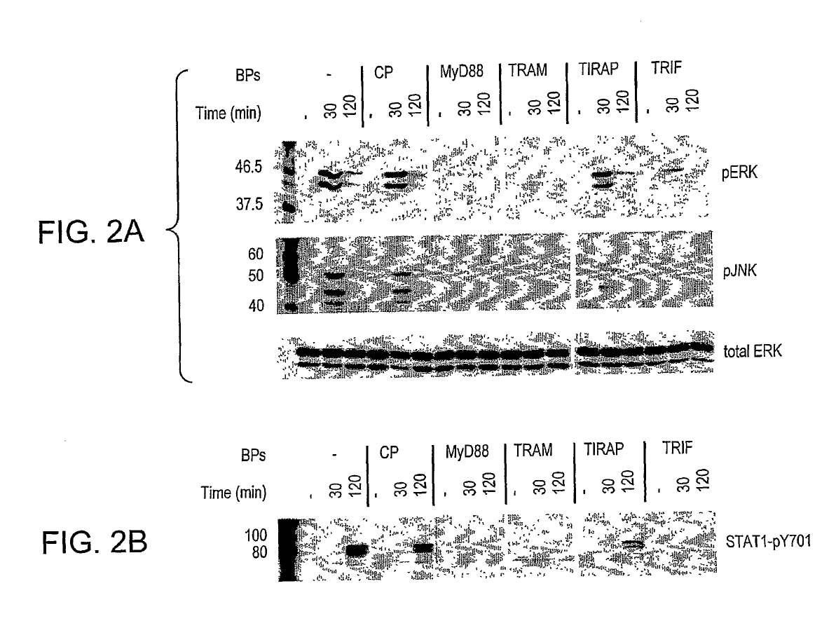

MAPKs regulate various cellular functions including signal transduction from TLRs and their activation can be detected significantly earlier than gene expression or cytokine secretion. Using antibodies specific for the phosphorylated (activated) forms of JNK, ERK, and p38, we next sought to examine the effect of BB-loop-containing inhibitory peptides on LPS-stimulated activation of MAPKs. LPS induces quick and transient activation of MAPKs in primary macrophages (FIG. 2A) that was not affected by the addition of CP. However, pre-incubation of cells for 1 h with TRAM or MyD88 BPs strongly blocked activation of ERK and JNK in response to LPS (FIG. 2A). Similar results were observed for activated p38 (data not shown). The TRIF BP also strongly inhibited phosphorylation of MAPKs, although inhibition of ERK activation was incomplete. The TIRAP BP was the weakest BP; all MAPKs were activated in the presence of it, although to a lesser extent when compared to CP.

### Example 3

Effect of BB-Loop Blocking Peptides on Activation of STAT-1 by LPS

Signal transducer and activator of transcription-1 (STAT-1) propagates signal transduction emanating from both type I and type II interferon receptors (reviewed in Horvath, C. M. 2000, Trends Biochem. Sci. 25, 496). Two phosphate acceptor sites are involved in activation of STAT-1. While phosphorylation of Tyr701 is critical for homo- or heterodimerization and nuclear translocation of STAT-1, phosphorylation of Ser727 modulates its transcriptional activity (reviewed in Horvath, C. M., 2000, supra). We previously reported that tyrosine phosphorylation of STAT1 by LPS depends on MyD88-independent induction of IFN-β and the subsequent autocrine activation of type I interferon receptors (Toshchakov et al., 2002, supra).

LPS-induced Tyr701 phosphorylation of STAT-1 was strongly inhibited by the TRAM and TRIF blocking peptides (FIG. 2B). Consistent with its strong ability to block IFN-β gene expression (FIG. 1), the MyD88 BP was also highly effective at inhibiting Tyr701 phosphorylation of STAT-1 (FIG. 2B). Treatment of macrophages with the TIRAP BP, however, did not completely block tyrosine phosphorylation of STAT-1 in response to LPS, although it was significantly diminished compared to the level seen in the presence of the CP (FIG. 2B). LPS-induced Ser727 phosphorylation of STAT-1 was similarly blocked by the BPs (data not shown). The BPs failed to inhibit IFN-β-induced Tyr701 phosphorylation of STAT-1 (data not shown), consistent with the observation that the type I IFN receptor does not depend on any of these adapters to signal (reviewed in Horvath C. M., 2000, supra).

### Example 4

Effect of BB-Loop Blocking Peptides on Activation of IRF-1 and IRF-3

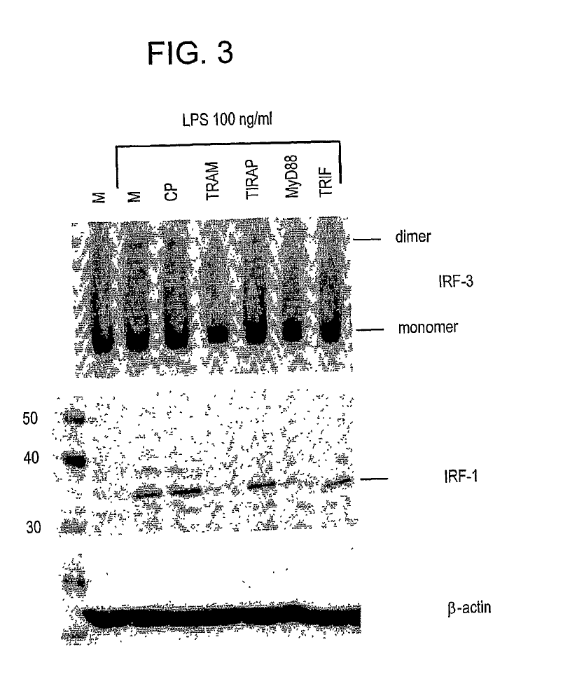

Interferon Regulatory Factors (IRFs) were first identified as important transcriptional mediators of the antiviral immune response (reviewed in Taniguchi et al., 2001, Annu. Rev. Immunol. 19, 623). Later, it was recognized that this family of transcription factors plays an essential role in signal transduction mediated by different TLRs. IRF-1 is regulated transcriptionally by signaling from different TLRs, as well as by cytokines induced by TLR engagement (e.g., IL-1, TNF, and IFN-β) (Fujita et al., 1989, Proc. Natl. Acad. Sci. USA 86, 9936). In contrast, IRF-3 is constitutively expressed in many cell types (Au et al., 1995, Proc. Natl. Acad. Sci. USA 92, 11675). Upon stimulation through TLR4 or TLR3, IRF-3 is phosphorylated at multiple sites in its C-terminus, and this modification enables it to dimerize and translocate to the nucleus where it targets IRF-3-responsive genes (Kawai et al., 2001, supra; Lin et al., 1999, Mol. Cell. Biol. 19, 2465). Using MyD88 knockout mice, Kawai et al. (Kawai et al., 2001, supra) demonstrated that LPS activates IRF-3 via the MyD88-independent pathway, and this, in turn, leads to the induction of IFN-β, the signature cytokine gene for the MyD88-independent pathway (Toshchakov et al., 2002, supra). Pretreatment of cells with TRAM, TRIF, or MyD88 BPs significantly inhibited LPS-induced dimerization of IRF-3 (FIG. 3). The TIRAP BP also decreased the amount of detectable IRF-3 dimer; however, inhibition was not as complete as with the other BPs.

IRF-1 is an immediate response gene, quickly induced by signaling from different TLRs, which contributes to the induction of other important TLR-inducible genes (e.g., iNOS). Importantly, MyD88 knockout macrophages are capable of inducing IRF-1 mRNA (Yamamoto et al., 2003, supra). Stimulation of macrophages by LPS for 1.5 h after pretreatment of cells with medium only or CP resulted in a strong upregulation of IRF-1 protein (FIG. 3). As was observed for IRF-3, TRAM and MyD88 peptides were the strongest inhibitors with respect to both IRF-3 and IRF-1. The TRIF BP again showed intermediate efficacy, while the TIRAP peptide was the weakest of the BPs.

### Example 5

### Limited Capacity of Adapter BB-Loop Inhibitory Peptides to Block TLR2-Induced Signaling

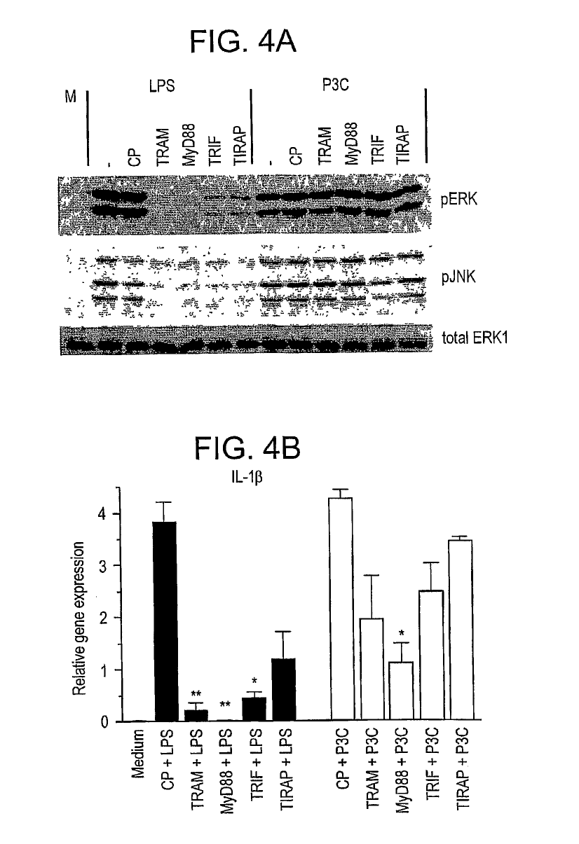

Apart from the use of a CP, we sought to analyze further the specificity of the inhibitory effects of BB-loop peptides on TLR signaling. Therefore, we also tested the effect of the peptides on signal transduction elicited from TLR2. TLR2 was chosen because it shares MyD88-dependent signaling pathway with TLR4, and TLR2-mediated responses are depreciated in both MyD88 and TIRAP-deficient cells (Yamamoto et al, 2002, supra; Horng et al., 2002, Nature 420, 329); however, TLR2-mediated signaling does not appear to utilize MyD88-independent arm for signal transduction (reviewed in Akira and Takeda, 2004, supra). Stimulation of MyD88 knockout macrophages with the Pam3Cys, a TLR2 agonist, does not activate MAPKs (Horng et al., 2002, supra; unpublished observation), whereas LPS-stimulated MyD88 knockout macrophages exhibit MAPKs and NF-κB activation, albeit with delayed kinetics (Kawai et al., 1999, supra). FIG. 4A demonstrates that the BPs differentially affect phosphorylation of ERK and JNK in response to LPS vs. Pam3Cys. Surprisingly, under conditions where LPS-induced MAPK phosphorylation was strongly blocked by all BPs, none inhibited Pam3Cys-induced phosphorylation of MAPKs.

The effect of BPs on Pam3Cys-induced expression of IL-1β mRNA is shown in FIG. 4B. LPS and Pam3Cys induced comparable levels of IL-1β mRNA, which were not significantly affected by treatment of cells with CP (not shown). Similar to their effect on activation of MAPKs, BPs were much weaker inhibitors with respect to Pam3Cys- vs. LPS-induced expression of IL-1β mRNA. Only the MyD88 BP exerted a statistically significant inhibitory effect (FIG. 4B) and this effect was considerably less than that seen in the case of LPS stimulation: in LPS-stimulated cells, MyD88 BP reduced IL-1β mRNA expression to <5% of the level detected in the presence of the CP, while in Pam3Cys-stimulated cells, IL-1β gene expression was reduced to approximately 25% of control levels. Taken collectively, these data demonstrate that TLR4-induced signaling is much more susceptible to inhibition by the adapter BB-loop BPs than is TLR2-mediated signaling.

### Example 6

### Mutations that Disrupt Function of Native TRAM do not Significantly Affect Function of Tram BB Construct

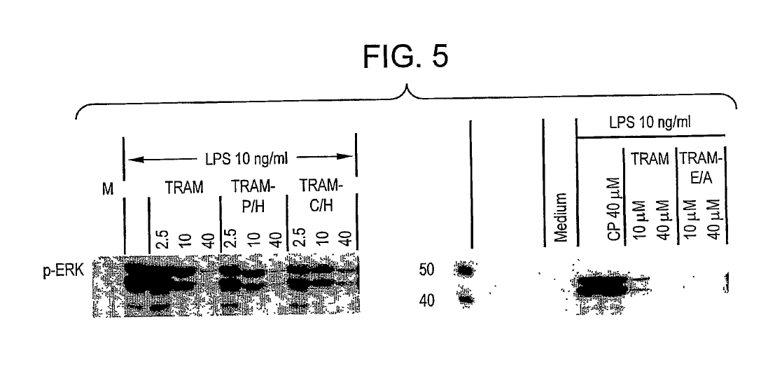

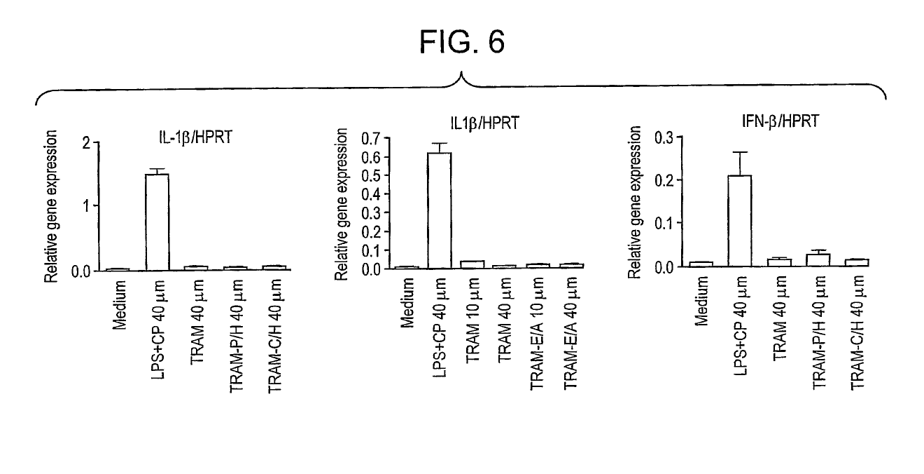

It was reported that introduction of mutations into the BB loop of TRAM, in particular substitutions of C117 or P116 for hystidine, disrupts function of this protein (Fitzgerald et al. 2003, supra). In this connection, we synthesized TRAM BB peptides that bear homologous mutations and checked functional properties of these constructs. To our surprise, inhibitory properties of these mutants were close to those of the native construct (FIGS. 5 and 6). Our current explanation for this observation is that the described mutations are substitutions for a larger amino acid that provides steric hindrance for interaction of BB loop with its target(s). Such mechanism works only when sequence of BB loop is incorporated into the stiff, three-dimensional structure of native protein globe. Our artificial constructs have free ends what allows for more flexibility in the peptide chain. This flexibility explains how the mechanism of ‘steric hindrance’ can be avoided.

In addition, we introduced E/A replacement in the position homologous to E114 of native TRAM. This replacement also did not affect considerably inhibitory capacity of the TRAM BB peptide (FIGS. 5 and 6). This finding suggests that the side chain of E114 is not critical for recognition/binding of TRAM and TRAM BB peptide to its target(s).

### Example 7

### DD Loop of MyD88 does not Provide Inhibitory Potential

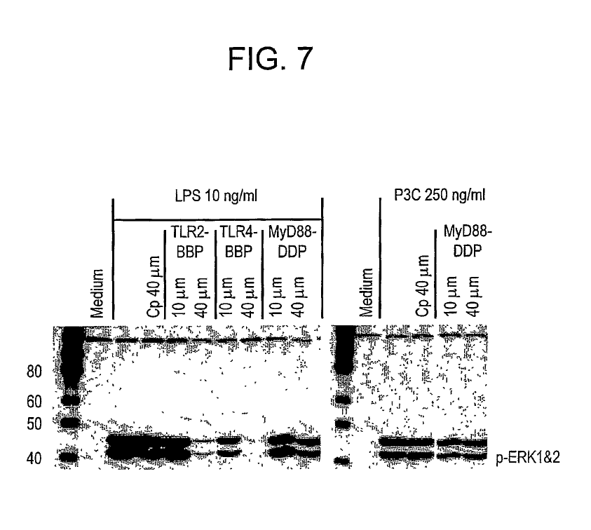

It is known that MyD88 is critical for TLR2-mediated signaling. On the other hand, Myd88 BB peptide is a poor inhibitor of P3C-induced signaling (Example 5). These facts taken together may suggest that surfaces of MyD88 other than that formed by its BB loop may be important for formation of TLR2 signaling platform. In this connection, we designed a penetrating peptide comprised of the DD loop of MyD88 and tested its effects on LPS- and P3C-induced activation of ERK and cytokine induction. MyD88 DD loop peptide was not inhibitory in respect to the parameters tested (FIG. 7). This finding shows the specificity of the inhibitory action of BB sequences. At the same time, it suggests that MyD88 DD peptide can be used as a mock, control sequence.

### Example 8

### TLR2 and TLR4 BB Peptides Inhibit Certain, not All Manifestations of TLR Signaling

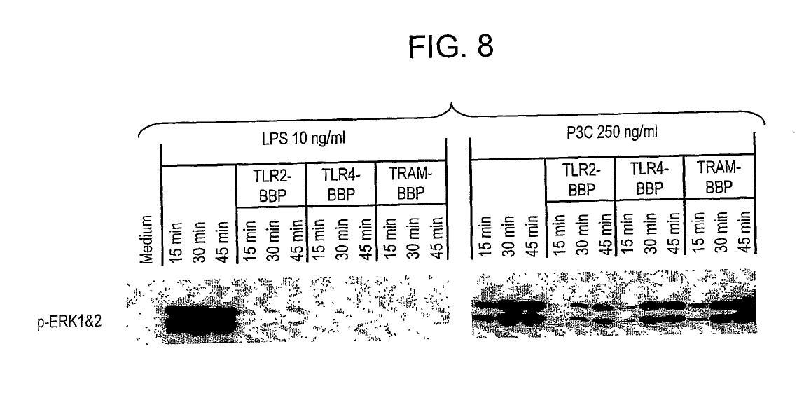

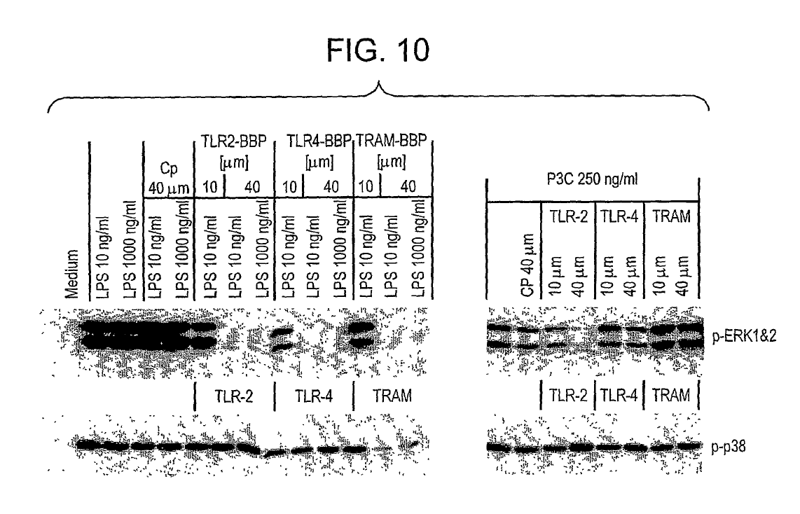

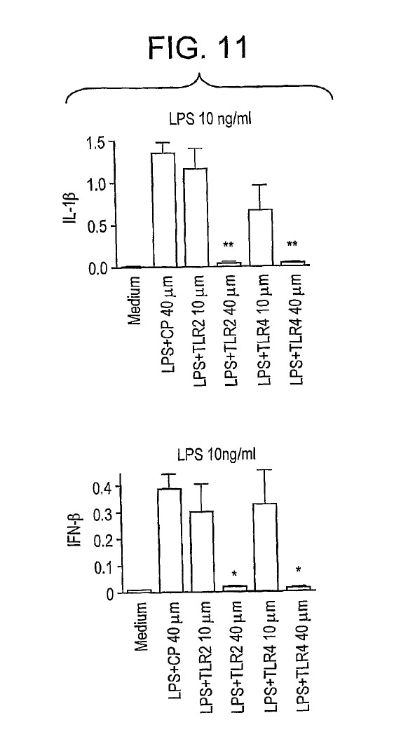

We have found that the inhibitory action of adapter BB peptides is considerably stronger towards TLR4-induced responses. One theoretical explanation for this phenomenon is that the adapter BB peptides preferentially bind and sequester this receptor. Next, we sought to design and examine functional properties of penetrating constructs made of homologous regions of TIR domains of several TLRs, TLR4, TLR2, and TLR1 and 6. TLR1 and TLR6 were chosen as both can heterodimerize with TLR2 in an agonist-dependent manner. Interestingly, sequences of BB loops of TLR1 and TLR6 are identical (Table 1). FIG. 8 shows that TLR2 and TLR4 BB peptides are potent inhibitors of LPS-induced activation of ERK. In addition, TLR2 and, to a lesser extend, TLR4 BB peptides also diminished and delayed phosphorylation of ERK induced by TLR2/TLR1 agonist, P3C (right panel of FIG. 8). Inhibitory action of these receptor BB peptides on a TLR2-induced response is at variance with previously described effects of adapter BB peptides which were highly specific for TLR4-induced signaling. Then, we examined effects of TLR2 and TLR4 BB peptides on a different MAP kinase, p38. It was a bigger surprise that these peptides did not inhibit LPS- or P3C-induced activation of p38 (FIG. 10). This finding shows that, unlike adapter peptides, TLR2 and TLR4 peptides are more selective towards separate pathways activated by a TLR and less discriminative in respect to receptor specificity. It also suggests that different pools of signaling molecules are sequestered by receptor vs. adapter peptides. We next examined the effects of TLR2 and TLR4 BB peptides on induction of IL1-β and IFN-β mRNA by LPS and P3C. Despite opposite effects of these receptor peptides on activation of ERK and p38, their effect on gene induction was inhibitory (FIG. 11 for LPS-induced gene induction; for P3C-induced cytokine induction data not shown).

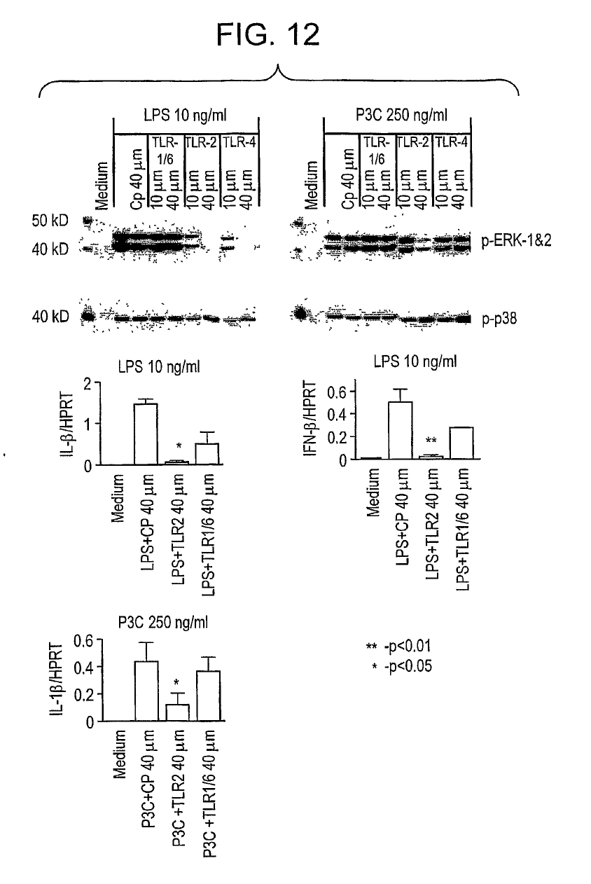

TLR1/6 receptor peptide affected neither LPS- or P3C-induced activation of MAP kinases, nor significantly affected induction of cytokines (FIG. 12). This finding suggests that this sequence can be used as a mock control for TLR2 and TLR4 BB peptides.

### Example 9

Penetrating BB Loop Peptides Inhibit TLR4 Signaling in a Non-Competitive Manner

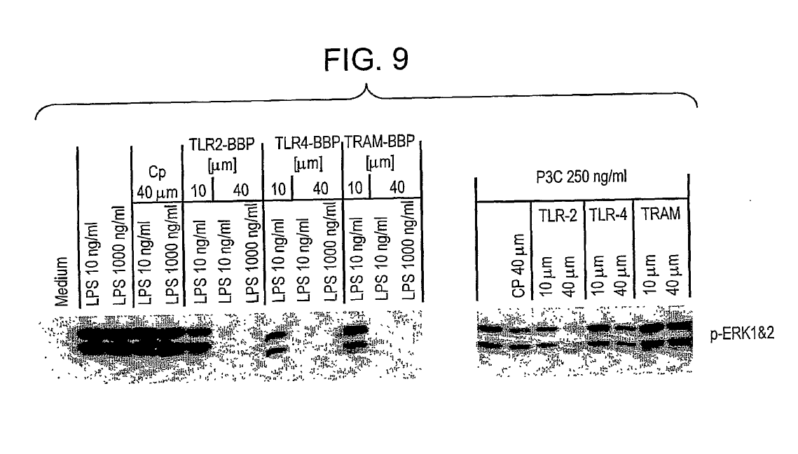

Several molecules of peptide nature can bind LPS directly and inhibit LPS-induced responses by this mechanism. Polymyxin B, Limulus endotoxin-binding protein-protease inhibitor (Minetti S. A. et al, 1991, J. Biol. Chem. 266, 20773-20780) are examples of these proteins. The feature typical for this mechanism is its competitive nature, i.e. excess LPS can overcome the inhibitory action of LPS-binding peptides. To exclude the possibility that the BB constructs exert their action via a similar mechanism, we examined whether the excess LPS can overcome the inhibitory effect of the BB peptides. Results of a representative experiment are shown in FIG. 9. A hundred-fold increase in concentration of LPS, from 10 ng/nl to 1 μg/ml, in the absence of a blocking peptide, did not result in further upregulation of ERK (FIG. 9). An increase in TLR2, TLR4, or TRAM blocking peptide concentration from 10 μM to 40 μM effectively inhibited phosphorylation of ERK, whether or not 100-fold excess LPS was used (FIG. 9). We observed a similar pattern of inhibition for MyD88 BB peptide (data not shown). These findings demonstrate the non-competitive mechanism of inhibition resulting from receptor and adapter BB peptides and suggest that LPS and blocking peptides act via non-overlapping sites.

### Discussion

This study represents an attempt to use penetrating oligopeptides containing homologous sequences that presumably interfere with the protein-protein interaction surfaces formed between the BB-loops of four adapter proteins with their target protein(s) to investigate mechanisms of signal transduction emanating from TLR4 and TLR2. The impetus for generating these four adapter BPs stemmed from contradictory results obtained using the TIRAP BP and a TIRAP dominant-negative mutant construct vs. macrophages from TIRAP knockout mice. The TIRAP BP was first used by Horng et al. (Horng et al., 2001, supra) as a complementary tool to confirm the involvement of this adapter in LPS-signaling and was reported to inhibit TLR4-, but not TLR9-mediated signaling (Horng et al., 2001, supra; Toshchakov et al., 2002, supra). However, using this same TIRAP BP, we found that it also inhibited LPS-induced IFN-β gene expression and IFN-β-luciferase reporter activity (Toshchakov et al., 2002, supra), while later studies using macrophages from TIRAP knockout mice found that induction of IRF-3 and IRF-3/STAT1-dependent genes by LPS was intact (Yamamoto et al., 2002, supra). Therefore, we sought to carry out a systematic comparison of the effects of BPs based on the homologous sequences of the other known TLR4 adapters to gain insights into the role of adapter BB-loops for protein interactions required for TLR4 signaling. Although the mechanism by which cell-penetrating, BB-loop peptides inhibit TLR signaling has not been formally demonstrated, it has been postulated that such peptides, once inside the cell, can occupy the docking site of the cognate adapter on its target and prevent binding of the native adapter. This, in turn, would be predicted to disrupt the formation of a functional signaling platform (Vogel et al., 2003, supra; Vogel and Fenton, 2003, Biochem. Soc. Trans. 31, 664).

Analysis of the effects of BPs on the expression of early-inducible genes revealed an interesting and completely unexpected pattern: the BPs showed little specificity with respect to genes induced by LPS via MyD88-dependent or -independent pathways. For example, the MyD88 peptide did not preferentially block IL-1β and MIP-1β, genes, which, according to data derived from knockout mice, require MyD88 for induction (Bjorkbacka et al., 2004, supra; Kawai et al., 1999, supra). Conversely, TRAM BP was equally effective in inhibiting both MyD88-dependent and -independent genes, in contrast to the conclusion drawn from experiments with TRAM knockout mice (Yamamoto et al., 2003, supra). The fact that all four BPs block

TLR4 signaling through either the MyD88-dependent or independent pathways suggests strongly that the stability of the TLR4 receptor signaling platform is indeed disrupted or fails to assemble correctly as a consequence of BP-target protein interactions.

In this regard, the disruption of TLR4 signaling by BPs is more reminiscent of a “receptor-knockout phenotype,” where both branches of the TLR4 signaling pathway are inhibited, rather than an “adapter-knockout” phenotype characterized by expression of distinct subsets of genes. The observation that BPs are capable of inhibiting various manifestations of LPS signaling suggests that they act before the signal bifurcates into separate pathways, most likely at the level of the receptor.

By examining events upstream of gene expression and cytokine secretion, it was possible to identify differences in the relative inhibitory capacities of BPs on TLR4 signaling. Thus, FIGS. 2 and 3 show that MyD88=TRAM>TRIF>TIRAP BPs with respect to inhibition of MAPK, STAT-1, or IRF activation. Various reasons can be invoked to explain the observed differences in the relative efficiency of individual BPs to inhibit LPS signaling.

Differences in peptide permeability, intracellular peptide turnover, existence of stable, “inefficient” conformations that do not bind as strongly to the intended molecular target, or, simply, non-specific binding to unrelated sites could all potentially contribute to our observations. Importantly, the efficiency of the peptides failed to correlate with their general physical-chemical parameters that are summarized in Table I.

In addition to the surprising result that all four BPs inhibited both MyD88-dependent and independent branches of TLR4-mediated signaling, the observations that the BPs were much weaker inhibitors of TLR2-mediated signaling was equally unexpected. The ability of BB-loop peptides to disrupt TLR4 signaling again supports the hypothesis that these regions of the adapters are critical for interaction with TLR4. The fact that the BPs did not affect TLR2-induced activation of JNK and ERK and exerted a much weaker effect on TLR2-induced IL-4 gene expression implies that the adapter BB-loops do not belong to the adapter surfaces that interact with TLR2. It is possible that the MyD88 BP partially interferes with the MyD88-TLR2 interaction surface, thus resulting in a lesser degree of inhibition. By this same logic, it would be predicted that TIRAP does not utilize its BB-loop in the formation of an active TLR2 signaling complex. These findings suggest that the architecture of receptor-adapter complexes differs between TLR2 and TLR4. The idea that adapters shared by different TLRs can interact with individual receptors in fundamentally different ways supports the existence of asymmetrical models of TIR-TIR complex formation. Interestingly, recent computer modeling performed by Dunne et al. (Dunne et al., 2003, supra) predicted that TLR4 and TLR2 bind TIRAP or MyD88 through different non-overlapping sites further support such a model.

1. An isolated blocking peptide comprising a BB-loop sequence of a Toll-like receptor-4 (TLR4) adapter protein.

2. The blocking peptide of claim 1 wherein said adapter is chosen from the group consisting of MyD88, said peptide having the sequence specified in SEQ ID NO:1, TRAM, said peptide having the sequence specified in SEQ ID: 2, TIRAP, said peptide haying the sequence identified in SEQ ID NO:3, and TRIF, said peptide having the sequence specified in SEQ ID NO:4.

3-5. (canceled)

6. The blocking peptide of claim 2, further comprising a cell-permeable peptide fused to said blocking peptide creating a blocking peptide fusion protein.

7. The blocking peptide fusion protein of claim 6 wherein said cell-permeable peptide is a cell-penetrating segment of antennapedia homeodomain, said blocking peptide fusion protein selected from the group consisting of MyD88 fusion peptide specified in SEQ ID NO:13, TRAM fusion peptide specified in SEQ ID NO:14, TIRAP fusion peptide specified in SEQ ID NO:15, and TRIF peptide specified in SEQ ID NO:16.

8. (canceled)

9. An isolated blocking peptide comprising a BB-loop sequence of a Toll-like receptor (TLR).

10. The blocking peptide of claim 9 wherein said blocking peptide is chosen from the group consisting essentially of TLR2 peptide having the sequence specified in SEQ ID NO:9, TLR4 peptide having the sequence specified in SEQ ID NO:10, and TLR1 or TLR6 peptide haying the sequence specified in SEQ ID NO:11.

11-12. (canceled)

13. The blocking peptides of claim 10, further comprising a cell-permeable peptide fused to said blocking peptide creating a blocking peptide fusion protein.

14. The blocking peptide fusion protein of claim 13 wherein said cell-permeable peptide is cell-penetrating segment of antennapedia homeodomain wherein said fusion proteins are selected from the group consisting essentially of TLR2 fusion peptide specified in SEQ ID NO:18, TLR4 fusion peptide specified in SEQ ID NO:19, TLR1/6 fusion peptide specified in SEQ ID NO:17.

15-18. (canceled)

19. A method for selectively disrupting MyD88-dependent and MyD88-independent signaling pathways of TLR4 comprising introducing into a cell a blocking peptide according to claim 1 in an amount sufficient to produce said disruption.

20. (canceled)

21. The method of claim 19 wherein said blocking peptides are selected from the group consisting of MyD88 (SEQ ID NO:1), TRAM (SEQ ID NO:2), TIRAP (SEQ ID NO:3), and TRIF (SEQ ID NO:4).

22. The method of claim 21 wherein the blocking peptides are fused to a cell-penetrating segment of antennapedia homeodomain creating fusion peptides selected from the group consisting of MyD88 (SEQ ID NO:13), TRAM (SEQ ID NO:14), TIRAP (SEQ ID NO:15), and TRIF (SEQ ID NO:16).

23-25. (canceled)

26. A method for selectively blocking ERK phosphorylation without affecting p38 phosphorylation comprising introducing into a cell any combination of blocking peptides according to claim 9 in an amount sufficient to produce said disruption.

27. The method of claim 26 wherein said blocking peptides are selected from the group consisting of TLR2 (SEQ ID NO:9) and TLR4 (SEQ ID NO:10).

28. The method of claim 27 wherein the blocking peptides are fused to a cell-penetrating segment of antennapedia homeodomain creating fusion peptides selected from the group consisting of TLR2 (SEQ ID NO:18) and TLR4 (SEQ ID NO:19).

29. A method for preventing an inflammatory response due to TLR4 activation in a host comprising administering a blocking peptide selected from the group consisting of (i) a BB-loop sequence from a TLR4 adapter protein selected from the group consisting of MyD88 (SEQ ID NO:1), TRAM (SEQ ID NO:2), TIRAP (SEQ ID NO:3), and TRIF (SEQ ID NO:4) and (ii) a Toll-like receptor BB-loop peptide selected from the group consisting of TLR2 (SEQ ID NO:9) and TLR4 (SEQ ID NO:10).

30. (canceled)

31. The method of claim 29 wherein said blocking peptide is fused to a cell-permeable sequence.

32. The method of claim 31 wherein said cell-permeable sequence is a cell penetrating segment of antennapedia homeodomain creating a fusion peptide selected from the group consisting of MyD88 (SEQ ID NO: 13), TRAM (SEQ ID NO: 14), TIRAP (SEQ ID NO: 15), TRIF (SEQ ID NO: 16), TLR2 (SEQ ID NO:18) and TLR4 (SEQ ID NO:20).

33. The method of claim 32 wherein said inflammatory response is associated with at least one of an allergy, asthma, contact dermatitis, delayed-type hypersensitivity, wound-healing, allergic rhinitis, food hypersensitivity, ectopic dermatitis, inflammatory bowel disease, an immunologic disease of the lung, an autoimmune or immune-mediated skin disease, psoriasis, gluten-sensitive enteropathy, rheumatoid arthritis, a graft rejection, sepsis, and septic shock.

34-36. (canceled)

37. The method of claim 31 wherein the blocking peptide is administered in combination with a compound used to treat or prevent any inflammation-related condition.

38-39. (canceled)

40. A kit for use in the method according to claim 31, said kit comprising at least one blocking peptide selected from the group consisting of MyD88 (SEQ ID NO:13), TRAM (SEQ ID NO:14), TIRAP (SEQ ID NO:15), TRIF (SEQ ID NO:16), TLR2 (SEQ ID NO:18), TLR4 (SEQ ID NO:19), TLR1/6 (SEQ ID NO:17) and control peptide (SEQ ID NO:20).
